(19) United States
(12) Patent Application Publication
(10) Pub. No.: US 2006/0173418 A1
(43) Pub. Date: Aug. 3, 2006

(54) Loss of resistance syringe

(75) Inventors: Dominic P. Rinaudo, Blandon, PA (US); Mark T. Salzberger, Sinking Spring, PA (US); Mark J. Spinka, Reading, PA (US)

Correspondence Address: AMSTER, ROTHSTEIN & EBENSTEIN LLP, 90 PARK AVENUE, NEW YORK, NY, 10016, US

(73) Assignee: ARROW INTERNATIONAL, INC.

(21) Appl. No.: 11/010,523

(22) Filed: Dec. 13, 2004

(51) Int. Cl. A61M 5/315

(52) U.S. Cl. 604/230

(57) ABSTRACT

A syringe including a barrel having an inner barrel portion and an outer barrel portion and a plunger slideably disposed within the barrel, the plunger having an inner plunger portion and an outer plunger portion. At least one of the inner barrel portion and the outer plunger portion is made of a low friction and high precision material, such as glass.

### TECHNICAL FIELD

The present invention is related to loss of resistance syringes.

### BACKGROUND

Analgesic or anesthetic drugs can be delivered to the spinal cord by placing the drugs outside of the membranous sac containing the spinal cord, which avoids unwanted side-effects of these drugs on the brain. Between this sac, called the dura, and the overlying spinal ligaments, is a potential space called the spinal epidural space (SES). It is a potential space because normally the anatomy here is juxtaposed until the space is crested. Placing drugs in the SES blocks spinal cord functions including pain transmission permitting either pain control (analgesia) or complete loss of all sensation (anesthesia) for surgery.

In clinical practice, locating the SES with a needle is technically difficult. The greatest danger for the novice is to sense the change in resistance as the needle passes through the spinal ligaments before the needle inadvertently passes through the SES and penetrates the dura. Penetration of the dura can lead to leakage of cerebral spinal fluid, the leakage being associated with problems such as post-dural puncture headache. Thus, it is most beneficial for clinicians to have the ability to determine the precise moment when the needle is advanced into the epidural space to decrease the likelihood of puncturing the dura.

The most commonly adopted method for determining entry into the epidural space is known as the “loss of resistance” technique. The loss of resistance technique involves insertion of the epidural needle through the skin into the interspinous ligament. Then, the stylet of the needle is removed and an air-tight and free sliding loss-of-resistance (LOR) syringe, containing air or saline, is connected to the needle. If the needle tip is properly positioned within the substance of the interspinous ligament, injection will not be possible; this is defined as the feeling of resistance. At this point, gentle but continuous pressure is applied to the plunger of the syringe. As the needle passes through the ligarnentum flavum and enters the epidural space, a sudden loss of resistance occurs. The medication can then be injected with precision into the epidural space.

Glass syringes are conventionally used in epidural anaesthesia employing the loss of resistance technique because the low friction between the plunger and the barrel allows the clinician to better sense the loss of resistance when the needle enters the epidural space. Such syringes include a generally cylindrical syringe barrel made of glass and a plunger made of a ground glass rod that closely fits within the cylinder. The glass syringes previously used have suffered from a number of disadvantages. They are expensive since the grinding requires close tolerances, on the order of 0.0007 inches clearance between the piston and the cylindrical syringe body. They are easily breakable, which poses a hazard to both patient and doctor. Also, the glass plunger and the glass barrel of each syringe must commonly be matched during the grinding by the manufacturer, since variations in grinding from one plunger to another may be sufficient to permit leakage of air or other material around the plunger. Thus, the barrels and plungers cannot easily be individually mass produced since the plungers often cannot be satisfactorily interchanged one with another in any given barrel. In addition, special metal holders for the glass barrel are often required to prevent the plunger from falling out of the barrel of its own weight. Further, glass syringes require metal tips through which the needle can extend, thereby increasing the overall cost and bulkiness of these syringes.

Attempts have been made to avoid these disadvantages by either manufacturing both the barrel and the plunger out of materials other than glass, such as plastics, or by using glass barrels with plastic plungers. The challenge has been to reduce the friction between a plastic barrel and a plastic plunger. One method to reduce this friction involves decreasing the amount of actual contact between these parts. For example, U.S. Pat. No. 4,354,507 (“the '507 Patent”) discloses a syringe in which both the barrel and the plunger are made of plastic. The plunger of the '507 Patent includes a compressible and elastomeric plunger tip having an annular wiper lip, the wiper lip being the only part of the plunger that engages the inner wall of the barrel so as to reduce frictional drag and permit the plunger to be easily moved axially in the barrel. However, although the reduced contact between the barrel and the plunger reduces frictional drag, it is not reduced to the level achieved in the case of syringes made completely of glass.

Other syringes, such as that disclosed in U.S. Pat. No. 6,171,286 (“the '286 Patent”), use a glass barrel and a plastic plunger, with a glass attachment on the plastic plunger that allows the plunger to move within the barrel with low resistance. The plastic plunger of the '286 Patent is partially disposed within a bore of a glass member, and the glass member slides in contact with the glass barrel. However, the glass barrel of the '286 Patent is still susceptible to the problems previously mentioned with respect to all-glass syringes, such as increased breakage.

Accordingly, there is a need for a syringe that provides sufficiently low resistance between the barrel and the plunger so as to be effective when used in the loss of resistance technique, while also avoiding the problems associated with conventional loss of resistance syringes, such as breakage and high cost.

### SUMMARY OF THE INVENTION

One aspect of the invention provides a syringe which exhibits reduced weight and cost while maintaining low frictional resistance between barrel and plunger portions of the syringe.

Another aspect of the invention provides a syringe that is useable in the loss of resistance technique, without requiring all-glass syringe components.

A syringe according to an exemplary embodiment of the invention includes a barrel having an inner barrel portion and an outer barrel portion and a plunger slideably disposed within the barrel, the plunger having an inner plunger portion and an outer plunger portion. At least one of the inner barrel portion and the outer plunger portion is made of glass.

In at least one embodiment, both the inner barrel portion and the outer plunger portion are made of glass.

In at least one embodiment, the plunger includes a distal end and a proximal end, and a glass sheath is formed over an entire length of the plunger, the glass sheath forming the outer plunger portion. The glass sheath may also be formed over the distal end of the plunger.

In at least one embodiment of the invention, a glass tip is formed at the distal end of the plunger, and the glass tip forms the outer plunger portion.

In at least one embodiment, at least one of the outer barrel portion and the inner plunger portion is made of plastic.

In at least one embodiment, both the outer barrel portion and the inner plunger portion are made of plastic.

A syringe according to another exemplary embodiment of the invention includes a hollow cylindrical barrel having an inner surface, and at least a portion of the inner surface of the barrel is made of glass, and an outer surface of the barrel is made of plastic. A cylindrical plunger is slideably disposed within the barrel, and at least a portion of the outer surface of the plunger is made of glass and a core portion of the plunger is made of plastic, such that the at least a portion of the outer surface of the plunger slides with substantially no frictional interference along the at least a portion of the inner surface of the barrel when the plunger is advanced inside the barrel.

A syringe according to another exemplary embodiment of the invention includes a barrel having an inner barrel portion and an outer barrel portion, and a plunger slideably disposed within the barrel. The plunger includes an inner plunger portion and an outer plunger portion, at least one of the inner barrel portion and the outer plunger portion being made of a high precision material. The coefficient of kinetic friction between the inner barrel portion and the outer plunger portion is less than about 0.40.

These and other features of this invention are described in, or are apparent from, the following detailed description of various exemplary embodiments of this invention.

### BRIEF DESCRIPTION OF THE DRAWINGS

Various exemplary embodiments of this invention will be described in detail, with reference to the following figures, wherein:

FIG. 1 is a perspective view of a syringe according to an exemplary embodiment of the invention;

FIG. 2 is a cross-sectional view of the syringe of FIG. 1; and

FIG. 3 is a cross-sectional view of a syringe according to another exemplary embodiment of the invention.

### DETAILED DESCRIPTION OF PREFERRED EMBODIMENTS

The various exemplary embodiments of the present invention are directed to a syringe including a plunger and a barrel, where the contacting surfaces between the plunger and the barrel are made of a low friction and high precision material, the material preferably being glass. The remaining portions of the plunger and barrel may be made of any other suitable material, preferably plastic. In at least one embodiment of the invention, at least a portion of the plunger is encased in a glass sheath and at least a portion of the inner surface of the barrel is made of glass, so that the core structure of the plunger and a substantial part of the barrel is made of a plastic material, and only those portions necessary to reduce frictional drag between the barrel and plunger are made of glass. The syringe according to the various exemplary embodiments of the invention is particularly useful as a loss of resistance syringe, i.e., a syringe used in performing the loss of resistance technique to deliver analgesic or anesthetic drugs directly to the spinal cord. However, it should be appreciated that the inventive concept is not limited as being useful only in the loss of resistance technique, but is instead applicable to any other medical procedure requiring a syringe.

In the present disclosure, like reference numbers refer to like elements throughout the drawings, which illustrate various exemplary embodiments of the invention.

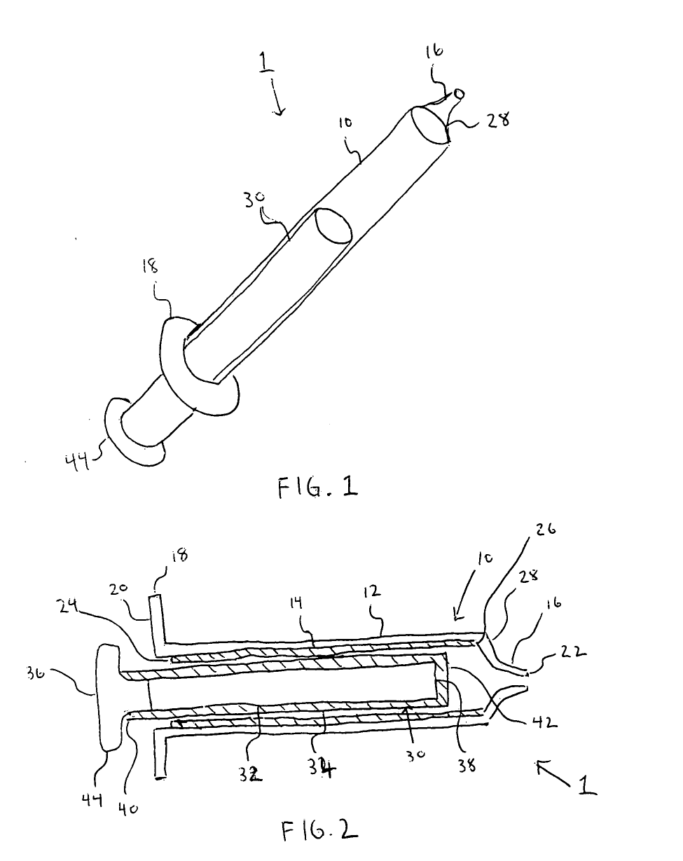

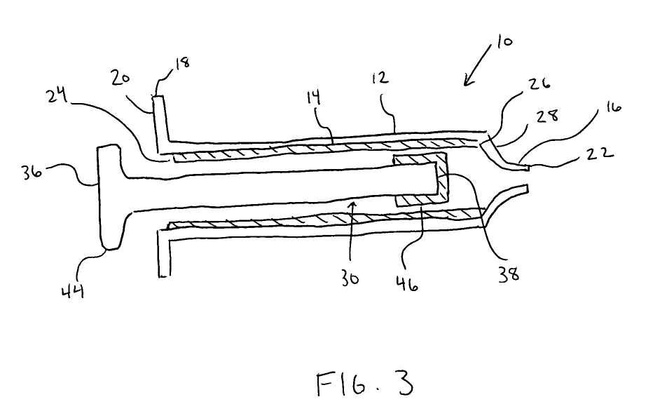

FIGS. 1 and 2 show a syringe 1 according to an exemplary embodiment of the invention. FIG. 1 is a perspective view of the syringe 1, and FIG. 2 is a cross-sectional view of the syringe 1.

The syringe 1 includes a barrel 10 and a plunger 30. As is conventional in the art, the overall diameter of the plunger 30 is smaller than that of the barrel 10 so that the plunger 30 can fit and slide within the barrel 10. The barrel 10 is composed of an outer barrel portion 12 and an inner barrel portion 14. The plunger is composed of an inner plunger portion 32 and an outer plunger portion 34.

The outer barrel portion 12 has a shape similar to that of a conventional syringe barrel, and has a proximal end 20 and a distal end 22. The outer barrel portion 12 includes a tip 16 disposed at the distal end 22, to which a needle (not shown) can be attached. More specifically, the outer barrel portion 12 includes a flange portion 28 disposed at the distal end 22 of the outer barrel portion 12, which terminates at the tip 16. A finger piece 18 is disposed at the distal end 20 of the outer barrel portion 12. The finger piece 18 generally has the shape of an annular flange, but can have any other suitable shape that provides support for two fingers, such as, for example, hexagonal. The outer barrel portion 12 can be made of any suitable material, preferably a molded polymeric material such as, for example, polyethylene or polypropylene.

The inner barrel portion 14 has a tube-like structure with a proximal end 24 and a distal end 26. The inner barrel portion 14 has a smaller length than that of the outer barrel portion 12. The distal end 26 of the inner barrel portion 14 preferably terminates at the beginning of the flange portion 28 of the outer barrel portion 12. Although the proximal end 24 of the inner barrel portion 14 is shown disposed inwards from the proximal end 20 of the outer barrel portion 12, the present invention is not limited to this construction. The inner barrel portion 14 is preferably made of glass, such as, for example, borosilicate glass. However, in other embodiments of the invention, the inner barrel portion 14 may be made of any other low friction and high-precision material that can be formed to tight tolerances, such as, for example, a high-precision plastic composite, ceramic, sapphire, quartz or metal. Preferably, the inner diameter of the inner barrel portion is formed with a dimensional tolerance of less than about +/− 1 micron. Also, the material used to form the inner barrel portion 14 is preferably transparent, although in other embodiments the material may be opaque.

The inner plunger portion 32 has a proximal end 36 and a distal end 38. An end cap 44 is disposed at the distal end 36 of the inner plunger portion 32, and may be molded as one piece with the inner plunger portion 32, or may be a separate piece attached to the inner plunger portion 32 by, for example, glue or any other suitable adhesive. As is known in the art, the end cap 44 allows the clinician to push against the plunger 30 with his/her thumb to inject, for example, analgesic or anesthetic drugs into the patient's body. The inner plunger portion 32 is made of any suitable material, and preferably a molded polymeric material such as, for example, polyethylene or polypropylene.

The outer plunger portion 34 includes a proximal end 40 and a distal end 42. As shown in FIG. 2, while the proximal end 40 of the outer plunger portion 34 is open to allow the end cap 44 to extend beyond the outer plunger portion 34, the distal end 42 of the outer plunger portion 34 is closed around the distal end 38 of the inner plunger portion 32. However, in other embodiments of the invention, the distal end 42 of the outer plunger portion 34 may be open, as well. The outer plunger portion 34 is preferably made of a glass materials, such as, for example, borosilicate. However, in other embodiments of the invention, the outer plunger portion 34 may be made of any other low friction and high-precision material that can be formed to tight tolerances, such as, for example, a high-precision plastic composite, ceramic, sapphire, quartz or metal. Also, the material used to form the outer plunger portion 34 is preferably transparent, although in other embodiments the material may be opaque. Preferably, the outer diameter of the outer plunger portion 34 is formed with a dimensional tolerance of +/− 1 micron. The coefficient of kinetic friction (μₖ) between the outer plunger portion 34 and the inner barrel portion 14 is preferably less than about 0.40. The clearance between the outer plunger portion 34 and the inner barrel portion 14 is preferably on the order of about 0.0007 inches.

It should be obvious from the above-described construction that a substantial part of the barrel 10 and plunger 30 is made of a non-glass material, e.g. plastic. Thus, the syringe according to the present embodiment is lighter and less expensive than the conventional all-glass syringe. Also, the tip 16 need not be made of metal, as in conventional all-glass LOR syringes, which not only contributes to the decreased weight and cost of the syringe according to the present embodiment, but also eases its manufacturing process. In addition, the mostly non-glass structure of the present syringe allows it to exhibit increased durability and resistance to breakage. At the same time, there is low friction between the barrel 10 and plunger 30 due to the low friction between the outer plunger portion 34 and the glass inner barrel portion 14. Thus, the syringe according to the present embodiment of the invention is able to provide the advantages of all-glass LOR syringes, while avoiding the drawbacks of such syringes.

The various exemplary embodiments of the present invention are meant to encompass any syringe structure in which only the surfaces of the barrel and the syringe in contact with one another are made of low friction and high precision materials, such as glass. Thus, for example, the glass outer portion of the plunger need not extend along the entire length of the plunger, but may be disposed only at one end of the plunger. FIG. 3 illustrates a syringe according to an exemplary embodiment of the invention which incorporates such a structure. In particular, FIG. 3 has substantially the same structure as that of the embodiment shown in FIGS. 1 and 2, with the exception of a glass tip 46 formed at the distal end 38 of the plunger 30 rather than the glass outer portion extending through the entire length of the plunger. The glass tip 46 effectively widens the diameter of the distal end 38 of the plunger 30, so that only the glass tip 46 is in contact with the glass inner barrel portion 14 as the plunger is slid within the barrel 10. The glass tip 46 is shown in FIG. 3 as a sheath formed only around the distal end 38 of the plunger 30, but may also be a solid piece of cylindrical glass attached to the distal end of the plunger.

The syringe according to various exemplary embodiments of the invention may be formed by conventional overmolding and insert molding processes. For example, a plastic core may be insert molded within a hollow glass cylinder to form a plastic plunger having an outer glass surface, and plastic may be overmolded a hollow glass cylinder to form a barrel having an inner glass surface and an outer plastic surface. Unlike the manufacture of glass syringes, the molding processes of the present invention allow the plastic outer surface of the barrel to be easily designed for improved ergonomics, such as by providing a relatively widened finger piece. Also, since the entire syringe is not made of glass, the glass parts can be more easily manufactured with tighter tolerances. For example, the inner diameter of the glass inner tube of the barrel can be made with relatively tighter tolerances, and the outer glass surface of the plunger can be made with tighter outer diameter surfaces, resulting in a syringe structure that exhibits improved fit and lower resistance.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A syringe comprising:
a barrel comprising an inner barrel portion and an outer barrel portion;
a plunger slideably disposed within the barrel, the plunger comprising an inner plunger portion and an outer plunger portion, at least one of the inner barrel portion and the outer plunger portion being made of glass.

2. The syringe of claim 1, wherein the inner barrel portion and the outer plunger portion are made of glass.

3. The syringe of claim 1, wherein the plunger comprises a distal end and a proximal end, and a glass sheath is formed over an entire length of the plunger, the glass sheath forming the outer plunger portion.

4. The syringe of claim 3, wherein the glass sheath is formed over the distal end of the plunger.

5. The syringe of claim 1, wherein the plunger comprises a distal end and a proximal end, and a glass tip is formed at the distal end of the plunger, the glass tip forming the outer plunger portion.

6. The syringe of claim 1, wherein at least one of the outer barrel portion and the inner plunger portion is made of plastic.

7. The syringe of claim 1, wherein the outer barrel portion and the inner plunger portion are made of plastic.

8. The syringe of claim 1, wherein the outer barrel portion further comprises a proximal end and a distal end, and a tip portion disposed at the distal end that supports a needle.

9. The syringe of claim 8, wherein the outer barrel portion further comprises a flange portion disposed at the distal end of the outer barrel portion that terminates in the tip portion.

10. The syringe of claim 8, wherein the outer barrel portion further comprises a finger piece disposed at the proximal end of the barrel.

11. The syringe of claim 10, wherein the finger piece is an annular flange.

12. The syringe of claim 9, wherein the inner barrel portion comprises a proximal end and a distal end, the distal end of the inner barrel portion terminating at the flange portion of the outer barrel portion.

13. A syringe comprising:
a hollow cylindrical barrel comprising an inner surface, at least a portion of the inner surface of the barrel being made of glass, and an outer surface of the barrel being made of plastic; and
a cylindrical plunger slideably disposed within the barrel, at least a portion of the outer surface of the plunger being made of glass and a core portion of the plunger being made of plastic, such that the at least a portion of the outer surface of the plunger slides with substantially no frictional interference along the at least a portion of the inner surface of the barrel when the plunger is advanced inside the barrel.

14. The syringe of claim 13, wherein the plunger comprises a proximal end and a distal end, and the at least a portion of the outer surface of the plunger comprises a glass tip formed at the distal end of the plunger.

15. The syringe of claim 13, wherein the plunger comprises a proximal end and a distal end, and the at least a portion of the outer surface of the plunger comprises a glass sheath that extends over the entire length of the core portion of the plunger.

16. A syringe comprising:
a barrel comprising an inner barrel portion and an outer barrel portion;
a plunger slideably disposed within the barrel, the plunger comprising an inner plunger portion and an outer plunger portion, at least one of the inner barrel portion and the outer plunger portion being made of a high precision material, the coefficient of kinetic friction between the inner barrel portion and the outer plunger portion being less than about 0.40.
